(19) United States
(12) Patent Application Publication
(10) Pub. No.: US 2023/0030974 A1
(43) Pub. Date: Feb. 2, 2023

(54) OIL-BASED COMPOSITION

(71) Applicant: SHISEIDO COMPANY, LTD., Tokyo (JP)

(72) Inventors: Saki KAMADA, Tokyo (JP); Keita NISHIDA, Tokyo (JP); Kianping GAN, Tokyo (JP)

(21) Appl. No.: 17/757,839

(22) PCT Filed: Dec. 16, 2020
(86) PCT No.: PCT/JP2020/046915

§ 371 Date: Jun. 22, 2022

(30) Foreign Application Priority Data
Dec. 27, 2019 (JP) 2019-239659

(51) Int. Cl. A61K 8/81; A61K 8/92; A61K 8/25; A61K 8/06

(52) U.S. Cl. CPC A61K 8/8152; A61K 8/92; A61K 8/25; A61K 8/064

(57) ABSTRACT

An oil-based composition includes: (A) polyoxyethylene (POE) hardened castor oil having an HLB of 12 or less; (B) an organic-modified clay mineral; and (C) polar oil.

### TECHNICAL FIELD

The present invention relates to an oil-based composition.

### BACKGROUND ART

An oil-based composition containing a large amount of polar oil is often used as an ingredient in a cream and the like, which is applied to the skin, such as a cosmetic or a quasi-drug (PTL 1). In products containing such an oil-based composition, cyclic silicone oils, such as decamethylcyclopentasiloxane, are used to enhance the stability and comfort of the product. Cyclic silicone oils have excellent properties such as heat resistance, cold resistance, chemical stability, water repellency, and low surface tension, and are widely used in many industrial fields such as cosmetics.

In recent years, there has been a movement to diversify the chemical substances used in cosmetics and the content thereof, based on an awareness of environmental problems.

### CITATION LIST

### Patent Literature

### [PTL 1]

Japanese Laid-Open Patent Publication No. H10-120525

### SUMMARY OF INVENTION

### Technical Problem

When the oil-based composition is used for a cosmetic, a quasi-drug, and the like, it may be more preferable when the content of the polar oil is increased.

Accordingly, it is an object of one aspect of the present invention to provide an oil-based composition containing a large amount of polar oil

### Solution to Problem

As a result of intensive research to solve the above problem by the inventors of the present invention, one aspect of the present invention is: an oil-based composition including (A) polyoxyethylene (POE) hardened castor oil having an HLB of 12 or less; (B) an organic-modified clay mineral; and (C) polar oil.

### Advantageous Effects of Invention

According to one aspect of the present invention, an oil-based composition containing a large amount of polar oil can be provided.

### DESCRIPTION OF EMBODIMENTS

One aspect of the present invention relates to an oil-based composition including (A) polyoxyethylene (POE) hardened castor oil having an HLB of 12 or less, (B) an organic-modified clay mineral, and (C) polar oil. Hereinafter, one aspect of the present invention will be described in detail. Embodiments of the present invention are not limited thereto.

### <(A) Polyoxyethylene (POE) Hardened Castor Oil Having an HLB of 12 or Less>

The POE hardened castor oil is a castor oil in which hydrogen and polyethylene glycol (PEG) are bonded, and is widely contained in surfactants. The POE hardened castor oil used in one aspect of the present invention has an HLB of 12 or less. The term “HLB” refers to a value of hydrophilic-lipophilic balance (HLB) that indicates the degree of affinity of surfactants for water and oil.

As the HLB value according to one aspect of the present invention, the value calculated by the formula represented by Formula 1 described below is used.

HLB=7+11.7·log(MW/MO) [Formula 1]

MW represents molecular weight of a hydrophilic group and MO represents molecular weight of a lipophilic group.

Examples of the POE hardened castor oil having an HLB of 12 or less include, but are not limited to, hardened castor oil (HLB=6), POE (30) hardened castor oil (HLB=11), POE (20) hardened castor oil monolaurate (HLB=8), POE (60) POE (15) hardened castor oil monoisostearate (HLB=6), POE (50) hardened castor oil monoisostearate (HLB=12), POE (30) hardened castor oil triisostearate (HLB=6), POE (60) hardened castor oil triisostearate (HLB=10), and the like.

Preferably, examples of the POE hardened castor oil ether include polypropylene glycol (PEG)-10 hardened castor oil, PEG-20 hardened castor oil, PEG-30 hardened castor oil, and the like.

Examples of the POE hardened castor oil ether monoisostearate ester include PEG-5 hardened castor oil isostearate, PEG-10 hardened castor oil isostearate, PEG-50 hardened castor oil isostearate, and the like.

Examples of the POE hardened castor oil triisostearate ester include PEG-5 hardened castor oil triisostearate, PEG-10 hardened castor oil triisostearate, PEG-20 hardened castor oil triisostearate, PEG-30 hardened castor oil triisostearate, PEG-40 hardened castor oil triisostearate, PEG-60 hardened castor oil triisostearate, and the like.

Further preferably, examples include POE hardened castor oil ether, POE hardened castor oil ether monoisostearate, POE hardened castor oil ether triisostearate, and the like.

The POE hardened castor oil described above may be used as (A) the polyoxyethylene (POE) hardened castor oil having an HLB of 12 or less. The POE hardened castor oil described above may be used alone or in combination of two or more.

(A) The polyoxyethylene (POE) hardened castor oil having an HLB of 12 or less may be contained at 0.01 to 50% by mass, 0.01 to 40% by mass, and more preferably 0.01 to 30% by mass, in the total amount of the oil-based composition of one aspect of the present invention.

By using (A) the polyoxyethylene (POE) hardened castor oil having an HLB of 12 or less in the present invention, a stable cream product can be provided without containing cyclic silicone oils. Also, excellent effects in terms of gelation performance, washability, and stability, can be exhibited.

### <(B) Organic-Modified Clay Mineral>

An organic-modified clay mineral is one of colloidal hydrous aluminum silicates with a layered structure used as an emulsifying aid. Examples of the organic-modified clay mineral according to one aspect of the present invention include disteardimonium hectorite, dimethylalkylammonium hectorite, benzyl dimethyl stearyl ammonium hectorite, distearyl dimethyl ammonium chloride-treated aluminum-magnesium silicate, and the like. Examples of commercial products in which the organic-modified clay mineral is used alone or in combination of two or more include BENTONE 27 (benzyl dimethyl stearyl ammonium chloride treated hectorite, manufactured by National Lead Co. and Rheox, Inc.), BENTONE 34 (Chemical name: quaternium-18 bentonite, manufactured by Rheox, Inc.), BENTONE 38 (distearyl dimethyl ammonium chloride-treated hectorite, manufactured by National Lead Co.), BENTONE 38V (quaternium-18 hectorite, manufactured by Rheox, Inc.), CLAYTONE-40 (manufactured by Southern Clay Products, Inc.), CLAYTONE SO (manufactured by Southern Clay Products, Inc.), and the like. Preferred examples include benzyl dimethyl stearyl ammonium hectorite, dimethyl distearyl ammonium hectorite, and the like, which are obtained by organically modifying hectorite. It is particularly preferred to use dimethyl distearyl ammonium hectorite. These may be contained alone or in combination of two or more.

When these organic-modified clay minerals are contained, the long-term stability of the oil-based composition is improved.

(B) The organic modified clay mineral is contained at 0.01% to 30% by mass, preferably 0.05% to 15% by mass, in the total amount of the oil-based composition of one aspect of the present invention. When the content is within the range described above, sufficient stability is imparted to the oil-based composition, a fresh tactile feel when used is obtained, and the usability of the oil-based composition, such as easiness to take by the finger and lightness of spreading on the skin of the oil-based composition, can be improved.

The ratio of (A) to (B) of the oil-based composition of one aspect of the present invention is preferably in a range of 0.2:1 to 2:1 by mass.

### <(C) Polar Oil>

(C) The polar oil is not particularly limited as long as it is usually used in cosmetics, pharmaceuticals, and foods. As an index indicating degree of polarity of organic compounds, IOB value (Inorganic/Organic Balance (inorganic value/organic value)) indicating ratio of inorganic value to organic value is used. Regarding the “inorganic value” and the “organic value” in the IOB value, the “inorganic value” and the “organic value” are set according to various atoms or functional groups. For example, the “organic value” is 20 for one carbon atom in a molecule, and the “inorganic value” is 100 for one hydroxyl group in a molecule. The IOB value of the organic compound may be calculated by integrating the “inorganic value” and the “organic value” described above of all the atoms and functional groups in the organic compound (see, for example, Fujita, Vol. 11, No. 10, pp. 719-725, 1957). The IOB value of the polar oil according to one aspect of the present invention is preferably, but not limited to, 0.05 to 0.80.

Examples of such polar oil include oils having an ester group and a polyoxyalkylene group continuously, such as cetyl octanate having an ester and a polyoxyalkylene group, and vegetable oils.

### <Ultraviolet Absorbing Polar Oil>

As such polar oil, (C) the polar oil according to one aspect of the present invention may contain polar oil having an ultraviolet absorbing function (hereinafter, may also be referred to as ultraviolet absorbing polar oil). The ultraviolet absorbing polar oil is not particularly limited. Examples of the ultraviolet absorbing polar oil include, benzoic acid derivatives, salicylic acid derivatives, cinnamic acid derivatives, dibenzoylmethane derivatives, β,β-diphenylacrylate derivatives, benzophenone derivatives, benzylidene camphor derivatives, phenylbenzimidazole derivatives, triazine derivatives, phenylbenzotriazole derivatives, anthranilic acid derivatives, imidazoline derivatives, benzalmalonate derivatives, 4,4-diarylbutadiene derivatives, and the like. Specifically, it is as follows.

Examples of the benzoic acid derivatives include ethyl p-aminobenzoate (PABA), ethyl-dihydroxypropyl PABA, ethylhexyl-dimethyl PABA, glyceryl PABA, PEG-25-PABA, diethylamino hydroxybenzoyl hexyl benzoate, and the like.

Examples of the salicylic acid derivatives include homosalate, ethylhexyl salicylate (octyl salicylate), dipropylene glycol salicylate, TEA salicylate, and the like.

Examples of the cinnamic acid derivatives include octyl methoxycinnamate or ethylhexyl methoxycinnamate, isopropyl methoxycinnamate, isoamyl methoxycinnamate, cinoxate, DEA methoxycinnamate, diisopropyl methylcinnamic acid, glyceryl-ethylhexanoate-dimethoxycinnamate, di-(2-ethylhexyl)-4′-methoxybenzal malonate, and the like.

Examples of the dibenzoylmethane derivatives include butyl methoxybenzoylmethane (4-tert-butyl-4′-methoxydibenzoylmethane), and the like.

Examples of the β,β-diphenylacrylate derivatives include octocrylene, and the like.

Examples of the benzophenone derivatives include benzophenone-1, benzophenone-2, benzophenone-3 or oxybenzone, benzophenon-4, benzophenon-5, benzophenon-6, benzophenon-8, benzophenon-9, benzophenon-12, and the like.

Examples of the triazine derivatives include anisotriazine (bis-ethylhexyloxyphenol methoxyphenyltriazine), ethylhexyltriazone, diethylhexylbutamide triazone, tris(diisobutyl-4′-aminobenzalmalonate)-s-triazine, and the like.

Examples of the phenylbenzotriazole derivatives include drometrizol trisiloxane, methylenebis(benzotriazolyltetramethylbutylphenol), and the like.

Examples of the anthranilic acid derivatives include menthyl anthranilate, and the like.

Examples of the imidazoline derivatives include ethylhexyl dimethoxybenzylidene dioxoimidazolidine propionate, and the like.

Examples of the benzalmalonate derivatives include a polyorganosiloxane having a benzalmalonate functional group, and the like.

Examples of the 4,4-diarylbutadiene derivatives include 1,1-dicarboxy(2,2′-dimethylpropyl)-4,4-diphenylbutadiene, and the like.

(C) The polar oil according to one aspect of the present invention may contain the ultraviolet absorbing polar oil described above alone or in combination of two or more. For example, as the ultraviolet absorbing polar oil, ethylhexyl methoxycinnamate, bis-ethylhexyloxyphenol methoxyphenyl triazine, oxybenzone-3, diethylamino hydroxybenzoyl hexyl benzoate, homosalate, ethylhexyl salicylate (octyl salicylate), butyl methoxydibenzoylmethane, octocrylene, anisotriazine (bis-ethylhexyloxyphenol methoxyphenyltriazine), and ethylhexyltriazone may be contained in combination.

As described above, it is preferable to use a combination of a plurality of ultraviolet absorbing polar oils including the derivatives derived from different precursor compounds. Accordingly, a wide range ultraviolet protection effect can be provided.

The oil-based composition according to one aspect of the present invention in which (C) the polar oil contains the ultraviolet absorbing polar oil, is preferably used for sunscreen cosmetics and quasi-drugs.

As described above, when (C) the polar oil contains the ultraviolet absorbing polar oil, either only the ultraviolet absorbing polar oil may be contained, or the ultraviolet absorbing polar oil and a polar oil that does not have an ultraviolet absorbing function may be contained in combination. Examples of the polar oil that does not have an ultraviolet absorbing function include ester oils, polyalkylene oxide derivative oils, and the like. Specifically, it is as follows.

Examples of the ester oils include pentaerythrityl tetraethylhexanoate, isononyl isononanoate, isopropyl myristate, cetyl 2-ethylhexanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxystearate, ethylene glycol di(2-ethylhexanoate), dipentaerythritol fatty acid ester, N-alkyl glycol monoisostearate, neopentyl glycol dicaprate, diisostearyl malate, glyceryl di-2-heptylundecanoate, trimethylolpropane tris(2-ethylhexanoate), trimethylolpropane triisostearate, pentaerythritol tetra(2-ethylhexanoate), glyceryl tri(2-ethylhexanoate) (triethylhexanoin), trimethylolpropane triisostearate, cetyl 2-ethylhexanoate, 2-ethylhexylpalmitate, diethylhexyl 2,6-naphthalate, benzoic acid (12 to 15 carbon atoms) alkyl, cetearyl isononanoate, tri (caprylic acid/capric acid) glycerin, (dicaprylic acid/capric acid) butylene glycol, glyceryl trimyristate, tri 2-heptylundecanoate glyceride, castic oil fatty acid methyl ester, oleyl oleate, cetosteryl alcohol acetoglyceride, 2-heptylundecyl palmitate, diisobutyl adipate, 2-octyldodecyl N-lauroyl-L-glutamate, di(2-heptylundecyl) adipate, ethyllaurate, di(2-ethylhexyl) sebacate, 2-hexyldecyl myristate, 2-hexyldecyl palmitate, 2-hexyldecyl adipate, diisopropyl sebacate, di-2-ethylhexyl succinate, ethyl acetate, butyl acetate, amyl acetate, triethyl citrate, tripropylene glycol dipivalate, and the like. In particular, pentaerythrityl tetraethylhexanoate is preferred.

In particular, examples of the fatty acid ester oils include glyceryl triisooctanoate, glyceryl triisopalmitate, octyl isopalmitate, isopropyl isostearate, isostearyl isostearate, isocetyl isostearate, hexyl isostearate, myristyl isostearate, isocetyl octanoate, cetyl isooctanoate, isostearyl octanoate, isodecyl isononanoate, octyldodecyl dimethyl octanoate, oleyl myristate, isostearyl erucate, isocetyl stearate, octyl stearate, isostearyl palmitate, isocetyl palmitate, octyl palmitate, isostearyl myristate, isocetyl myristate, octyldodecyl myristate, decyl myristate, isopropyl palmitate, isopropyl myristate, trimethylolpropane triisostearate, neopentyl glycol dicaprate, dioctyl sebacate, octyldodecyl 12-stearoyl oxystearate, dioctyldodecyl stearoyl glutamate, dioctyl adipate, diisostearyl malate, octyldodecyl lactate, isostearyl lactate, octyl p-dimethylaminobenzoate, and the like.

Examples of the polyalkylene oxide derivative oils include diethylene glycol monopropyl ether, polyoxyethylene polyoxypropylene pentaerythritol ether, polyoxypropylene butyl ether, and the like.

As (C) the polar oil according to one aspect of the present invention, the ester oil, the polyalkylene oxide derivative oil, and the like described above may be contained either alone or in combination of two or more. When the polar oil containing the ester oil is used in combination with the ultraviolet absorbing polar oil, the dissolution of the solid ultraviolet absorbing polar oil is promoted.

The content of (C) the polar oil is 0.01° to 90% by mass, preferably 1% to 70% by mass, and even more preferably 5% to 50% by mass, in the total amount of the oil-based composition of one aspect of the present invention.

### <(D) Oil>

The oil phase of the oil-based composition according to one aspect of the present invention may contain oils other than (C) the polar oil. Examples of such oils include hydrocarbon oils, higher fatty acids, higher alcohols, silicone oils other than cyclic silicone oils, vegetable oils, animal oils, liquid oils, solid fats, waxes, perfumes, and the like used in typical oil-based compositions.

Specifically, examples of the hydrocarbon oils include undecane, tridecane, hydrogenated polydecene, isododecane, isohexadecane, isoparaffin, liquid paraffin, ozokerite, squalane, pristane, liquid paraffin, isoparaffin, alpha-olefin oligomer, polybutene, ceresin, squalene, Vaseline, microcrystalline wax, and the like. Among these, undecane, tridecane, hydrogenated polydecene, and isododecane are preferred because powder, particularly titanium oxide, can be well dispersed and the tactile feel can be improved.

Examples of the higher fatty acids include lauric acid, myristic acid, palmitic acid, stearic acid, behenic acid, oleic acid, undecylenic acid, tall acid, isostearic acid, linoleic acid, linolenic acid, eicosapentaenoic acid (EPA), docosahexaenoic acid (DHA), and the like. Among these, isostearic acid is preferred.

Examples of the higher alcohols include straight chain alcohols (for example, lauryl alcohol, cetyl alcohol, stearyl alcohol, behenyl alcohol, myristyl alcohol, oleyl alcohol, cetostearyl alcohol, and the like) and branched chain alcohols (for example, monostearylglycerin ether (batyl alcohol)-2-decyltetradecinol, lanolin alcohol, cholesterol, phytosterol, hexyldodecanol, isostearyl alcohol, octyldodecanol, and the like).

Examples of the silicone oils include dimethicone, caprylyl methicone, methylphenyl polysiloxane, methylhydrogen polysiloxane, decamethyl polysiloxane, dodecamethyl polysiloxane, tetramethyltetrahydrogen polysiloxane, diphenylsiloxy phenyl trimethicone, highly polymerized methyl polysiloxane, amino acid modified silicone, and the like. In particular, dimethicone, caprylyl methicone, and diphenylsiloxy phenyl trimethicone are preferred.

Preferably, the silicone oil does not contain the cyclic silicone oil. In one aspect of the present invention, the oil-based composition that does not contain cyclic silicones can be provided.

The oil-based composition according to one aspect of the present invention can provide an oil-based composition containing a large amount of polar oil, high stability and an excellent feeling when used is provided, even when cyclic silicones are not contained.

Examples of the vegetable oils include avocado oil, camellia oil, macadamia nut oil, corn oil, olive oil, rapeseed oil, sesame oil, persic oil, wheat germ oil, camellia kissi seed oil, castor oil, linseed oil, safflower oil, cottonseed oil, perilla oil, soybean oil, peanut oil, tea seed kaya oil, rice bran oil, palm kernel oil, tung jojoba oil, germ oil, and the like.

Examples of the animal oils include squalane, turtle oil, mink oil, egg yolk oil, and the like.

These materials can be contained alone or in combination of two or more, as oils according to one aspect of the present invention.

(D) The oil may be contained in the oil other than (C) the polar oil in an amount of 90% or more, in the oil-based composition according to one aspect of the present invention.

The ratio of the content of (B) the organic-modified clay mineral to the total content of (C) the polar oil and (D) the oil (the content of (B) the organic-modified clay mineral:(the content of (C) the polar oil the content of (D) the oil)) may be in a range of 1:3 to 1:100, and preferably 1:10 to 1:50.

### <(E) Silicone Surfactant>

The oil-based composition according to one aspect of the present invention may further include a silicone surfactant. A silicone surfactant is a polymer of an organosilicon compound having a surface-active action, and is highly water-repellent. The silicone surfactant is widely used as a film-forming agent in cosmetics, such as foundation, sunscreen, makeup bases, and conditioning agents for hair treatment.

Examples of the silicone surfactant suitable for one aspect of the present invention include, but are not limited to, (PEG-10/lauryl dimethicone) crosspolymer, (PEG-15/lauryl dimethicone) crosspolymer, (dimethicone/(PEG-10/15)) crosspolymer, PEG/PPG-20/22 butyl ether dimethicone, PEG-10 dimethicone, PEG-11 methyl ether dimethicone, PEG-3 dimethicone, PEG-9 dimethicone, PEG-9 methyl ether dimethicone, polyglyceryl-3 disiloxane dimethicone, lauryl PEG-9 polydimethylsiloxyethyl dimethicone, lauryl polyglyceryl-3 polydimethylsiloxyethyl dimethicone, and the like.

### <Wax>

Wax may be contained, as the oil according to one aspect of the present invention. As the wax suitable for one aspect of the present invention, waxes used for cosmetics and the like containing an oil-based composition can be used. Specifically, examples of the wax being waxes and paraffin waxes include Japan wax, hydrogenated castor oil, beeswax, candelilla wax, cotton wax, carnauba wax, bayberry wax, tree wax, spermaceti, montan wax, rice bran wax, kapok wax, sugarcane wax, jojoba wax, shellac wax, microcrystalline wax, ceresin, and paraffin.

Examples of the higher alcohols include behenyl alcohol, stearyl alcohol, cetyl alcohol, isostearyl alcohol, lauryl alcohol, myristyl alcohol, batyl alcohol, octyldodecanol, and the like.

Examples of the higher fatty acids include behenic acid, stearic acid, myristic acid, and the like.

Examples of the solid fats include cacao oil, coconut oil, horse tallow, hardened palm oil, palm oil, beef tallow, mutton tallow, hardened beef tallow, palm kernel oil, pork tallow, beef bone tallow, Japan wax kernel oil, hardened oil, beef foot oil, hexyl laurate, dextrin palmitate, Vaseline, polyethylene, batyl alcohol, and the like.

Examples of cholesterol esters include cholesterol, phytosterols, cholesteryl 12-hydroxystearate, cholesteryl lanolate, cholesteryl isostearate, cholesteryl ricinoleate, macadamia nut oil fatty acid cholesteryl ester, and the like.

Examples of phytosterol esters include phytosteryl 12-hydroxystearate, phytosteryl lanolate, phytosteryl isostearate, phytosteryl ricinoleate, macadamia nut oil fatty acid phytosteryl ester, and the like.

Examples further include lanolin, lanolin alcohol, hydrogenated lanolin alcohol, lanolin fatty acid, dipentaerythrite such as dipentaerythrityl hexa(hydroxystearate/stearate/rosinate) and dipentaerythrityl (hydroxystearate/isostearate), di(cholesteryl or phytosteryl/behenyl/octyldodecyl) N-lauroyl-L-glutamate, di(cholesteryl or phytosteryl/octyldodecyl)N-lauroyl-L-glutamate, and the like.

These waxes may be contained alone or in combination of two or more.

When waxes are contained in oil-based cosmetics and the like, application texture may be heavier and feeling of use may deteriorate, compared to a case of water-based products. That is, the content of the wax is preferably as low as possible. Thus, when the wax is contained in the oil-based composition according to one aspect of the present invention, the content thereof is 5% by mass or less, preferably 3% by mass or less, and even more preferably 2% by mass or less, in the total amount of the oil-based composition according to one aspect of the present invention.

### <Water-In-Oil Type Emulsion Composition>

The oil-based composition according to one aspect of the present invention may be a configuration of a water-in-oil type emulsion composition. When the water-in-oil type emulsion composition is used as a cosmetic, a moisturizing feeling after application is maintained and water retention is excellent as compared with aqueous cosmetics. Therefore, the water-in-oil type emulsion composition is suitably used as a cosmetic or a quasi-drug, for example, as a sunscreen emulsion or a sunscreen cream, applied to the skin of humans or animals, that maintains a moisturizing feeling after application and has excellent water retention properties. The water-in-oil type emulsion composition is also widely applicable to a variety of cosmetics requiring sunscreen effects, such as make-up and hair products, and the like.

When the oil-based composition according to one aspect of the present invention is used as the water-in-oil type emulsion composition, the size of the emulsified particles is preferably 1.0 to 20.0 μm in average particle size and 0.1 to 30.0 μm in particle size distribution, preferably 1.0 to 10.0 μm in average particle size and 0.1 to 20.0 μm in particle size distribution. When the size of the emulsified particle is within these ranges, a feeling when used such as a refreshing feeling is enhanced and good stability is maintained.

When the oil-based composition according to one aspect of the present invention is used as a water-in-oil type emulsion composition, an aqueous phase is formed in addition to the ingredients (A) to (C) described above. Aqueous ingredients of the aqueous phase are not particularly limited as long as they are aqueous ingredients commonly used in external compositions such as cosmetics, quasi-drugs, and the like. As the aqueous ingredients, water, water-soluble alcohol, a thicker, a moisturizing agent, a chelating agent, preservative, dye, and the like may be used.

Examples of the water include, purified water, ion-exchanged water, tap water, and the like. The content of the water may be appropriately selected depending on the use of the cosmetics or the quasi-drugs containing the water-in-oil type emulsion composition.

The surfactant may be a cationic surfactant, an anionic surfactant, a nonionic surfactant, an amphoteric surfactant, and the like. Among these, the nonionic surfactant is preferably used. The surfactant ingredient functions as an emulsifier or a gelling agent, and can improve the stability of the water-in-oil form. In particular, PEG-10 dimethicone is preferred as an emulsifying surfactant, and PEG-10 hydrogenated castor oil is preferred as a gelling surfactant.

The content of the surfactant is preferably 0.5% to 10% by mass, and more preferably 1% to 5% by mass, with respect to the total amount of the water-in-oil type composition.

As a configuration of the water-in-oil type emulsion composition according to one aspect of the present invention, the aqueous phase is stably present as emulsified particles in the oil-based composition. Cosmetics and the like having such a configuration can provide a good texture of spreading on the skin. The aqueous phase may also contain an aqueous phase ingredient including a substance compatible with water, a substance that dissolves in water, and the like, as described below.

The cosmetics are not particularly limited. Examples of the cosmetics include any cosmetics for skin care (also referred to as basic cosmetics) such as lotion, serum, emulsion, cream, and the like, and cosmetics for makeup (also referred to as make-up cosmetics) such as foundation, blusher, lip rouge, gloss, eyebrow cosmetics, eyeliner, eye shadow, mascara, manicure, perfume, and the like. In the present embodiment, preferably, the cosmetics are makeup bases and sunscreen.

One aspect of the present invention may be an external composition including the oil-based composition and the water-in-oil type emulsion composition. The external composition may be used, for example, as cosmetics such as sunscreen, scalp hair cosmetics, and the like, or as skin external agents, that may be used in the outer skin of humans or animals by application and the like. The external composition may take any product form, and may be used, for example, as an external composition for face, body, or scalp hair, such as lotion, emulsion, cream, facial mask, and the like.

“Application” means applying (or attaching by applying) cosmetics, external compositions, and the like.

### <Other Ingredients>

In addition to the ingredients (A) to (E) described above, the oil-based composition and/or the water-in-oil type emulsion composition according to one aspect of the present invention may contain, as other ingredients, moisturizers, ultraviolet absorbers, ultraviolet scattering agents, antioxidants, antimicrobial preservatives, vitamins, hormones, arginine, amino acids, anti-inflammatory agents, antimicrobial agents, whitening agents, astringents, refreshing agents, thickeners, sterols, alcohols, polymer substances, water, natural extracts extracted with aqueous alcohols and the like, dyes, neutralizing agents, pH adjusting agents, and the like.

Specifically, examples of the moisturizers include glycerin, 1,3-butylene glycol, polyethylene glycol, sorbitol, xylitol, maltitol, and the like.

In addition to the above-described ultraviolet absorbing polar oil as the polar oil, any commonly used ultraviolet absorber may be used. Specific examples include sodium hydroxymethoxybenzophenone sulfonate, sodium dihydroxydimethoxybenzophenone disulfonate, hydroxymethoxybenzophenone sulfonic acid and its trihydrate, sodium hydroxymethoxybenzophenone sulfonate, phenylbenzimidazole sulfonic acid, ferulic acid, terephthalylidene dicamphor sulfonic acid, benzylidene camphor sulfonic acid, and the like.

When the oil-based composition according to one aspect of the present invention is used as sunscreen, an ultraviolet scattering agent may also be contained in addition to the above-described ultraviolet absorber. As the ultraviolet scattering agent, any powder particles having an ultraviolet scattering effect may be used. For example, particulate titanium oxide, particulate zinc oxide, and the like may be used.

Examples of the whitening agents include tranexamic acid, potassium 4-methoxysalicylate, nicotinic acid amide, and the like.

Examples of the antioxidants include butyl hydroxytoluene, tocopherols, phytic acid, ascorbic acid, sodium pyrosulfite, and the like.

Examples of the antimicrobial preservatives include benzoic acid, sodium benzoate, salicylic acid, sorbic acid, alkylesters of parahydroxybenzoic acid (ethyl paraben, butyl paraben, and the like), methyl paraben, sodium dehydroacetate, phenoxyethanol, hexachlorophene, and the like.

Examples of the chelating agent include salicylic acid, edetic acid, metaphosphoric acid, and the like.

As various active ingredients, vitamins, hormones, arginine, amino acids, anti-inflammatory agents, antimicrobial agents, astringents, refreshing agents, sterols, and the like may be contained. Also, extracts derived from animals, plants, or microorganisms may be contained.

Specifically, examples include: vitamins such as vitamin A and its derivatives; vitamin B such as vitamin B6 hydrochloride, vitamin B6 tripalmitate, vitamin B6 dioctanoate, vitamin B2 and its derivatives, vitamin B12, vitamin B15 and its derivatives, and the like; vitamin C such as ascorbic acid, ascorbic acid sulfate ester (salt), ascorbic acid phosphate ester (salt), ascorbic acid dipalmitate, and the like; vitamin E such as α-tocopherol, β-tocopherol, γ-tocopherol, vitamin E-acetate, vitamin E-nicotinate, and the like; vitamin D; vitamin H; pantothenic acid; and pantothetine; nicotinic acid amide, benzyl nicotinate, γ-olizanol, allantoin, glycyrrhizic acid (salt), glycyrrhetinic acid and its derivatives, hinokitiol, mucidin, bisabolol, eucalyptol, thymol inositol, pantothenyl ethylether, ethinyl estradiol, cepharanthine, placenta extract, and the like.

Examples of the alcohol containing monohydric alcohol include ethanol, propanol, isopropanol, isobutyl alcohol, t-butyl alcohol, and the like.

Examples of the polymer substances include: plant-based polymers such as polyhydric alcohol, gum arabic, tragacanth gum, galactan, guar gum, carob gum, gum karaya, gellan gum, and carrageenan; microbial polymers such as xanthan gum, dextran, succinoglycan, pullulan, and sodium biohyaluronate; animal-based polymers such as collagen, casein, and gelatin; starch-based polymers such as carboxymethyl starch, and methyl hydroxypropyl starch; cellulose-based polymers such as methyl cellulose, ethyl cellulose, methyl hydroxypropyl cellulose, hydroxy ethyl cellulose, sodium cellulose sulfate, hydroxypropyl cellulose, carboxymethyl cellulose sodium, and crystalline cellulose; alginic acid-based polymers such as sodium alginate, and propylene glycol alginate; vinyl-based polymers such as polyvinyl alcohol, polyvinyl acetate, polyvinyl methyl ether, polyvinylpyrrolidone, copolymers of vinylpyrrolidone and vinyl acetate, and carboxy vinyl polymers; acrylic-based polymers such as sodium polyacrylate, polyethyl acrylate, alkanolamine polyacrylate, copolymers of alkyl methacrylate and dimethylamino ethyl methacrylate, poly 2-acrylamido-2-methylpropanesulfonic acid, and polymethacryloyloxytrimethyl ammonium; and polymers including 2-methacryloyloxyethyl phosphorylcholine as a monomer.

Examples of other ingredients further include acyl sarcosinate such as sodium lauroylsarcosinate, glutathione; a-hydroxy acids such as aminocaproic acid, citric acid, malic acid, lactic acid, glycolic acid, and tartaric acid, and their salts such as sodium and potassium, Rumex japonicus, Sophora flavescens, Nuphar japonica, orange, sage, yarrow, mallow, Cnidium, Swertia japonica, thyme, Angelica acutiloba, spruce, birch, field horsetail, sponge gourd, horse-chestnut, saxifrage, blackberry lily, chamomile, arnica, lily, mugwort, Chinese peony, aloe, gardenia, Chamaecyparis pisifera, royal jelly, placental extract, pantothenyl ethyl ether, dipotassium glycyrrhizinate, biotin, pyridoxine hydrochloride, adenosine triphosphate, α-lipoic acid, 3-O-ethylascorbic acid, magnesium ascorbyl phosphate, albutin, glucosyl hesperidin, glucosylrutin, dimethyl polysiloxanes such as decamethyl tetrasiloxane, dodecamethyl pentasiloxane, tetradecamethyl hexasiloxane, and hexadecamethyl heptasiloxane; and cyclic polysiloxanes such as octamethylcyclotetrasiloxane, decamethylcyclopentasiloxane, dodecamethylcyclohexasiloxane, and tetradecamethylcycloheptasiloxane.

These substances may be contained within a range that does not depart from the spirit of the present invention.

### <Method for Producing Oil-Based Composition>

The oil-based composition according to one aspect of the present invention may be produced by publicly-known methods. For example, as an example of a method for producing, the above-described aqueous ingredients such as water and the above-described suitable oily ingredients may be prepared using an emulsion device and the like. The method is not limited thereto, and a method for producing according to the properties of the target product and the like may be used.

The oil-based composition according to one aspect of the present invention is suitable for use in cosmetics, quasi-drugs, and the like, which contains a large amount of polar oil.

### EXAMPLE

The following are examples of the oil-based composition according to one aspect of the present invention. Those skilled in the art will understand that the substances, formulations, ratios, methods, and the like used in the present example can be appropriately changed without departing from the spirit of the present invention. Also, the description of the present example is not intended to limit the present invention to the scope of the example. In the following examples and the like, “%” regarding the content represents “% by mass” unless otherwise specified.

The compositions of the oil-based compositions used in the Examples and Comparative Examples are described in Tables 1 to 5. Of these, in Examples 1 to 16 and Comparative Examples 1 to 10, an oil-based composition was used. In Examples 17 to 19 and Comparative Example 11, an emulsion composition including the oil-based composition was used.

### Examples 1 to 16 and Comparative Examples 1 to 10

Oil-based compositions having the compositions described in the following tables were prepared according to conventional procedures, and stability of the oil-based compositions was evaluated as gel forming ability as follows.

Stability of the oil-based composition is achieved by promoting gelation of clay by combining surfactant and clay mineral contained in the oil-based composition. Therefore, a combination of proportions of surfactant and clay mineral that efficiently forms gel of the clay mineral was examined in the following examples.

### <Gel Forming Performance Test>

50 ml of the oil-based composition is placed in a screw tube (50 ml), and the state of the oil-based composition when tilted is visually observed. At this time, when the oil-based composition did not flow even when the screw tube was tilted, it was determined that gelation occurred and gel was formed. When the oil-based composition flowed when the screw tube was tilted, it was determined that gelation did not occur and gel was not formed.

A: Gel was formed.
B: Gel was not formed.

The results are described in Tables 1 to 5 below.

TABLE 1

| | | EXAM- | EXAM- | EXAM- | EXAM- | EXAM- | EXAM- | EXAM- | EXAM- |
|---|---|---|---|---|---|---|---|---|---|
| | INGREDIENT | PLE 1 | PLE 2 | PLE 3 | PLE 4 | PLE 5 | PLE 6 | PLE 7 | PLE 8 |
| OIL | ISODODECANE | 63.5 | 63.5 | 63.5 | 63.5 | 63.5 | 63.5 | 63.5 | 63.5 |
| ULTRAVIOLET | ETHYLHEXYL | 23.8 | 23.8 | 23.8 | 23.8 | 23.8 | 23.8 | 23.8 | 23.8 |
| ABSORBING | METHOXYCINNAMATE | | | | | | | | |
| POLAR OIL | | | | | | | | | |
| ORGANIC- | DISTEARDIMONIUM | 6.35 | 6.35 | 6.35 | 6.35 | 6.35 | 6.35 | 6.35 | 6.35 |
| MODIFIED | HECTORITE | | | | | | | | |
| CLAY MINERAL | | | | | | | | | |
| SURFACTANT | PEG-10 HYDROGENATED | 6.35 | | | | | | | |
| | CASTOR OIL | | | | | | | | |
| | PEG-20 HYDROGENATED | | 6.35 | | | | | | |
| | CASTOR OIL | | | | | | | | |
| | PEG-30 HYDROGENATED | | | 6.35 | | | | | |
| | CASTOR OIL | | | | | | | | |
| | PEG-5 HYDROGENATED | | | | 6.35 | | | | |
| | CASTOR OIL | | | | | | | | |
| | TRIISOSTEARATE | | | | | | | | |
| | PEG-10 HYDROGENATED | | | | | 6.35 | | | |
| | CASTOR OIL | | | | | | | | |
| | TRIISOSTEARATE | | | | | | | | |
| | PEG-20 HYDROGENATED | | | | | | 6.35 | | |
| | CASTOR OIL | | | | | | | | |
| | TRIISOSTEARATE | | | | | | | | |
| | PEG-30 HYDROGENATED | | | | | | | 6.35 | |
| | CASTOR OIL | | | | | | | | |
| | TRIISOSTEARATE | | | | | | | | |
| | PEG-40 HYDROGENATED | | | | | | | | 6.35 |
| | CASTOR OIL | | | | | | | | |
| | TRIISOSTEARATE | | | | | | | | |
| | TOTAL (%) | 100 | 100 | 100 | 100 | 100 | 100 | 100 | 100 |
| | GELATION ABILITY | A | A | A | A | A | A | A | A |

TABLE 2

| | | EXAM- | EXAM- | EXAM- | EXAM- | EXAM- | EXAM- | EXAM- | EXAM- |
|---|---|---|---|---|---|---|---|---|---|
| | INGREDIENT | PLE 9 | PLE 10 | PLE 11 | PLE 12 | PLE 13 | PLE 14 | PLE 15 | PLE 16 |
| OIL | ISODODECANE | 63.5 | 63.5 | 63.5 | 63.5 | | | | |
| | UNDECANE (65%)/ | | | | | 63.5 | | | |
| | TRIDECANE (35%) | | | | | | | | |
| | HYDROGENATED | | | | | | 63.5 | | |
| | POLYDECENE | | | | | | | | |
| | DIMETHICONE | | | | | | | 63.5 | |
| | CAPRYLYL METHICONE | | | | | | | | 63.5 |
| ULTRAVIOLET | ETHYLHEXYL | 23.8 | 23.8 | 23.8 | 23.8 | 23.8 | 23.8 | 23.8 | 23.8 |
| ABSORBING | METHOXYCINNAMATE | | | | | | | | |
| POLAR OIL | | | | | | | | | |
| ORGANIC- | DISTEARDIMONIUM | 6.35 | 6.35 | 6.35 | 6.35 | 6.35 | 6.35 | 6.35 | 6.35 |
| MODIFIED | HECTORITE | | | | | | | | |
| CLAY MINERAL | | | | | | | | | |
| SURFACTANT | PEG-20 HYDROGENATED | | | | | 6.35 | 6.35 | 6.35 | 6.35 |
| | CASTOR OIL | | | | | | | | |
| | PEG-60 HYDROGENATED | 6.35 | | | | | | | |
| | CASTOR OIL | | | | | | | | |
| | TRIISOSTEARATE | | | | | | | | |
| | PEG-5 HYDROGENATED | | 6.35 | | | | | | |
| | CASTOR OIL | | | | | | | | |
| | ISOSTEARATE | | | | | | | | |
| | PEG-10 HYDROGENATED | | | 6.35 | | | | | |
| | CASTOR OIL | | | | | | | | |
| | ISOSTEARATE | | | | | | | | |
| | PEG-50 HYDROGENATED | | | | 6.35 | | | | |
| | CASTOR OIL | | | | | | | | |
| | ISOSTEARATE | | | | | | | | |
| | TOTAL (%) | 100 | 100 | 100 | 100 | 100 | 100 | 100 | 100 |
| | GELATION ABILITY | A | A | A | A | A | A | A | A |

TABLE 3

| | | COMPAR- | COMPAR- | COMPAR- | COMPAR- | COMPAR- |
|---|---|---|---|---|---|---|
| | | ATIVE | ATIVE | ATIVE | ATIVE | ATIVE |
| | INGREDIENT | EXAMPLE 1 | EXAMPLE 2 | EXAMPLE 3 | EXAMPLE 4 | EXAMPLE 5 |
| OIL | ISODODECANE | 63.5 | 63.5 | 63.5 | 63.5 | 63.5 |
| ULTRAVIOLET | ETHYLHEXYL | 23.8 | 23.8 | 23.8 | 23.8 | 23.8 |
| ABSORBING | METHOXYCINNAMATE | | | | | |
| POLAR OIL | | | | | | |
| ORGANIC- | DISTEARDIMONIUM | 6.35 | 6.35 | 6.35 | 6.35 | 6.35 |
| MODIFIED | HECTORITE | | | | | |
| CLAY MINERAL | | | | | | |
| SURFACTANT | PEG-40 HYDROGENATED | 6.35 | | | | |
| | CASTOR OIL | | | | | |
| | PEG-10 GLYCERYL | | 6.35 | | | |
| | TRIISOSTEARATE | | | | | |
| | PEG-30 GLYCERYL | | | 6.35 | | |
| | TRIISOSTEARATE | | | | | |
| | PEG-3 GLYCERYL | | | | 6.35 | |
| | ISOSTEARATE | | | | | |
| | PEG-8 GLYCERYL | | | | | 6.35 |
| | ISOSTEARATE | | | | | |
| | TOTAL (%) | 100 | 100 | 100 | 100 | 100 |
| | GELATION ABILITY | B | B | B | B | B |

TABLE 4

| | | COMPAR- | COMPAR- | COMPAR- | COMPAR- | COMPAR- |
|---|---|---|---|---|---|---|
| | | ATIVE | ATIVE | ATIVE | ATIVE | ATIVE |
| | INGREDIENT | EXAMPLE 6 | EXAMPLE 7 | EXAMPLE 8 | EXAMPLE 9 | EXAMPLE 10 |
| OIL | ISODODECANE | 63.5 | 63.5 | 63.5 | 63.5 | 63.5 |
| ULTRAVIOLET | ETHYLHEXYL | 23.8 | 23.8 | 23.8 | 23.8 | 23.8 |
| ABSORBING | METHOXYCINNAMATE | | | | | |
| POLAR OIL | | | | | | |
| ORGANIC- | DISTEARDIMON1UM | 6.35 | 6.35 | 6.35 | 6.35 | 6.35 |
| MODIFIED | HECTORITE | | | | | |
| CLAY MINERAL | | | | | | |
| SURFACTANT | PEG-10 SOY STEROL | 6.35 | | | | |
| | PEG-8 DIISOSTEARATE | | 6.35 | | | |
| | BEHENETH-10 | | | 6.35 | | |
| | SORBITAN STEARATE | | | | 6.35 | |
| | DIGLYCERYL | | | | | 6.35 |
| | DIISOSTEARATE | | | | | |
| | TOTAL (%) | 100 | 100 | 100 | 100 | 100 |
| | GELATION ABILITY | B | B | B | B | B |

### Examples 17 to 19 and Comparative Examples 11

Emulsion compositions including an oil-based composition having the composition described in the following table were prepared according to conventional procedures, and stability of the emulsion compositions was evaluated as follows.

### <Stability Test>

The emulsion compositions obtained above were stored at room temperature for 1 month, then presence or absence of a change in appearance was visually examined and judged according to the following criteria.

A: The appearance did not change.
B: The appearance has changed.

TABLE 5

| | | | | | COMPAR- |
|---|---|---|---|---|---|
| | | | | | ATIVE |
| | | EXAM- | EXAM- | EXAM- | EXAMPLE |
| | INGREDIENT | PLE 17 | PLE 18 | PLE 19 | 11 |
| OIL | ISODODECANE | 20 | | 5 | 20 |
| | CAPRYLYL METHICONE | | 20 | | |
| | HYDROGENATED POLYDECENE | 3 | 3 | 18 | 3 |
| | DIPHENYLSILOXY PHENYL | 1.2 | 1.2 | 1.2 | 1.2 |
| | TRIMETHICONE | | | | |
| | ISOSTEARIC ACID | 0.5 | 0.5 | 0.5 | 0.5 |
| ULTRAVIOLET | ETHYLHEXYL METHOXYCINNAMATE | 5 | 5 | 5 | 5 |
| ABSORBING | | | | | |
| POLAR OIL | | | | | |
| POLAR OIL | PENTAERYTHRITYL | 3 | 3 | 3 | 3 |
| | TETRAETHYLHEXANOATE | | | | |
| EMULSIFYING | PEG-10 DIMETHICONE | 3.5 | 3.5 | 3.5 | 3.5 |
| SURFACTANT | | | | | |
| GELLING | PEG-10 HYDROGENATED | 1.3 | 1.3 | 1.3 | |
| SURFACTANT | CASTOR OIL | | | | |
| ORGANIC- | DISTEARDIMONIUM HECTORITE | 1.6 | 1.6 | 1.6 | 1.6 |
| MODIFIED | | | | | |
| CLAY MINERAL | | | | | |
| POWDER | HYDROPHOBIZED TITANIUM OXIDE | 8.9 | 8.9 | 8.9 | 8.9 |
| | METHYL METHACRYLATE | 4 | 4 | 4 | 4 |
| | CROSSPOLYMER | | | | |
| | HYDROPHOBIZED TALC | 1 | 1 | 1 | 1 |
| AQUEOUS | WATER | 33.8 | 33.8 | 33.8 | 35.1 |
| INGREDIENT | EDTA-3Na | 0.2 | 0.2 | 0.2 | 0.2 |
| | GLYCERIN | 2.5 | 2.5 | 2.5 | 2.5 |
| | DPG | 7 | 7 | 7 | 7 |
| | BG | 2 | 2 | 2 | 2 |
| | ETHANOL | 1 | 1 | 1 | 1 |
| | PHENOXYETHANOL | 0.5 | 0.5 | 0.5 | 0.5 |
| | TOTAL (%) | 100 | 100 | 100 | 100 |
| | STABILITY | A | A | A | B |

The present application claims the priority to Japanese Patent Application No. 2019-239659, filed on Dec. 27, 2019, with the Japanese Patent Office, the entire contents of which are hereby incorporated by reference.

1. An oil-based composition comprising:
(A) polyoxyethylene (POE) hardened castor oil having a hydrophilic-lipophilic balance (HLB) of 12 or less;
(B) an organic-modified clay mineral; and
(C) polar oil.

2. The oil-based composition according to claim 1, wherein the polyoxyethylene (POE) hardened castor oil having the HLB of 12 or less is at least one selected from the group consisting of POE hardened castor oil ether, POE hardened castor oil ether monoisostearate ester, and POE hardened castor oil triisostearate ester.

3. The oil-based composition according to claim 1, wherein the polar oil includes an ultraviolet absorber.

4. The oil-based composition according to claim 1, wherein a ratio of the polyoxyethylene (POE) hardened castor oil having an HLB of 12 or less to the organic-modified clay mineral is in a range of 0.2:1 to 2:1 by mass.

5. The oil-based composition according to claim 1, further comprising oil, wherein the oil is contained in oil other than the polar oil in an amount of 90% by mass or more.

6. The oil-based composition according to claim 1, further comprising a silicone surfactant.

7. The oil-based composition according to claim 5, wherein a ratio of a content of the organic-modified clay mineral to a total content of the polar oil and the oil is in a range of 1:3 to 1:100.

8. The oil-based composition according to claim 1, wherein in a case where the oil includes wax, a content of the wax is 2% by mass or less in a total amount of the oil-based composition.

9. The oil-based composition according to claim 1, wherein the oil-based composition is a water-in-oil type emulsion composition.
